(19) United States
(12) Patent Application Publication
(10) Pub. No.: US 2010/0130621 A1
(43) Pub. Date: May 27, 2010

(54) USE OF HYDROXYTYROSOL AS ANTI-AGING AGENT

(75) Inventors: Daniel Raederstorff, Flaxlanden (FR); Ying Wang-Schmidt, Stallikon (CH); Karin Wertz, Rheinfelden (DE)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/595,984

(22) PCT Filed: Apr. 17, 2008
(86) PCT No.: PCT/EP2008/003089

§ 371 Date: Oct. 15, 2009

(30) Foreign Application Priority Data
Apr. 18, 2007 (EP) 07007873.8

(51) Int. Cl. A61K 31/05; A61P 39/06; A61P 27/16; A61P 19/10; A61P 9/00; C07C 39/11

(52) U.S. Cl. 514/731; 568/763

(57) ABSTRACT

The present invention is directed to the use of (a composition comprising) hydroxytyrosol as anti-aging agent. The composition, to which the present invention is also directed, does essentially not comprise resveratrol and is administered orally to animals. The present invention is further directed to anti-aging methods. “Anti-aging” meaning in the context of the present invention: retarding the aging processes in said animals, improving age-related physiological deficits in said animals and/or promoting a healthy aging in said animals.

### FIELD OF THE INVENTION

The present invention is directed to the use of hydroxytyrosol and compositions comprising hydroxytyrosol as an anti-aging agent. The present invention is further directed to anti-aging methods. “Anti-aging” in the context of the present invention refers to retarding the aging processes in animals, improving age-related physiological deficits in animals and/or promoting a healthy aging in animals.

### BACKGROUND OF THE INVENTION

Mitochondria are organelles in the cell responsible for energy production. The mitochondrial inner membrane is embedded with a respiratory chain containing complexes I, II, III, IV and V, which transport electrons and produces ATP via a series of redox reactions, a process called oxidative phosphorylation.

In addition to their well known function of supplying energy to a cell, mitochondria and their components participate in a number of other cellular activities. For example, mitochondria also control thermogenesis and the apoptosis process and are thus involved in the ageing process.

The mitochondria contain a high level of oxidants, since the respiratory chain generates reactive species, e.g. superoxide anions, if it works with reduced efficiency or during energy uncoupling. Superoxide anions are generated as by products in several steps of electron transport chain, such as the reduction of coenzyme Q in complex III, where a highly reactive free radical is formed as an intermediate (Q.—). This unstable intermediate can lead to electron “leakage”, when electrons jump directly to oxygen and form the superoxide anion, instead of moving through the normal series of well-controlled reactions of the electron transport chain.

An antioxidant is a molecule capable of slowing or preventing the oxidation of other molecules. Antioxidants terminate oxidation chain reactions by removing free radical intermediates, and inhibit other oxidation reactions by being oxidized themselves. Reducing agents such as thiols or polyphenols often exert antioxidant property. Well known antioxidants such as Vitamin A, C and E scavenge free radicals and protect DNA, proteins and lipids from damage. Antioxidants also protect mitochondria from reactive oxygen species and free radicals generated during ATP production.

While it has been generally accepted in the past that administration of antioxidants would be beneficial to promote mitochondrial biogenesis, this has not been shown to be the case. Gomez-Carbera et al. 2008 Am. J Clin. Nutr. 87(1):142-149, demonstrated in a double-blinded randomized clinical study, that oral administration of 1 g Vitamin C per day decreases mitochondrial biogenesis in skeletal muscle.

Hydroxytyrosol has been described in the past as having positive cardiovascular effects (see, e.g. Gonzalez-Santiago et al 2006 Atherosclerosis 188:35-42; or Mitro et al 2003 NMCD. Nutritional Metabolism and Cardiovascular Diseases 13(5):306; but these are concerned with the anti-atherosclerotic effects of hydroxytyrosol and/or its status as an antioxidant.

The instant invention is directed to hydroxytyrosol's anti-ageing properties which are distinct from its ability to act as an antioxidant

### DETAILED DESCRIPTION OF THE INVENTION

This invention pertains to the use of a composition comprising hydroxytyrosol as anti-aging agent, wherein the composition does essentially not comprise resveratrol and wherein the composition is administered orally to animals.

Aging is characterized by a progressive loss in physiological functions that is probably caused by accumulated damage in a variety of cellular components. Mitochondria are ubiquitous organelles generating energy in cells by converting nutrients in adenosine triphosphate (ATP) molecules which are used for normal cell functioning and maintenance. Mitochondria are also involved in regulating cell survival. Recently it was suggested that loss of mitochondrial function not only contribute to diseases but also plays an important role in the aging process. A decrease in mitochondria number in certain organs and an impairment of the mitochondrial respiratory chain is often associated with the aging process and is considered as a major contributor to aging. Healthy subjects age 65-75 year show signs of altered mitochondrial properties characterized by a loss of oxidative enzyme activity and tissue mitochondrial content. Moreover, tissues obtained from aged animals display changes in mitochondrial structure associated with a decreased energy production. Aged human and animals organs have increased levels of mitochondrial DNA (mtDNA) mutations in tissues and mtDNA damage is inversely correlated to maximal life span. Calorie restriction without malnutrition which is the best recognized method to increase life span also increase genes encoding for proteins involved in mitochondrial function in human skeletal muscle and reduce DNA damage indicative of cell aging. Thus, aging is associated with reduced mitochondrial biogenesis and an accumulation of mitochondrial damage.

“Mitochondrial biogenesis” refers to processes of growth, amplification and healthy maintenance of the mitochondria. Mitochondrial biogenesis is a complex process involving both nuclear and mitochondrial players. Mitochondrial DNA encodes a small number of proteins, which are translated on mitochondrial ribosomes. Most of these proteins are highly hydrophobic subunits of the respiratory chain, which is localized in the inner mitochondrial membrane. Nuclear-encoded proteins are translated on cytosolic ribosomes and imported into mitochondria. These proteins include structural proteins, enzymes or enzyme subunits, components of the import-, replication-, transcription- and translation-machinery and chaperones.

The peroxisome proliferator-activated receptor-γ coavtivator-1 (PGC-1) is a co-transcriptional regulation factor of cellular energy metabolism which is involved in the control of mitochondrial function and induces mitochondrial biogenesis. The decrease of PGC-1 in aging tissues is a key factor in mitochondrial dysfunction which may be prevented by an elevation of PGC1 leading to an increase mitochondrial biogenesis. Hydroxytyrosol improves mitochondrial function through an activation of the mitochondrial respiratory chain complexes and an increase in mitochondrial biogenesis. Thus, an improvement in mitochondrial function could prevent cellular aging and consequently aging of the body. Therefore, hydroxytyrosol can be considered as an useful agent to prevent aging and age-related diseases.

### BRIEF DESCRIPTION OF THE FIGURES

FIG. 1 shows expression of PGC-1α. A) Western blot analysis in adipocytes of PGC-1α. B) Quantitative values tabulated for PGC-1α:α-tubulin ratio with a densitometry. Values are mean±SE of five experiments. * p<0.05 vs. control; **P<0.01 vs.control.

FIG. 2 shows expression of mitochondrial proteins. 3T3-L1 adipocytes were treated for 48 hrs with hydroxytyrosol. Cells were subsequently solubilized into SDS sample buffer and analyzed by Western blotting with antibodies against α-tubulin, mitochondrial electron transport complex I, complex II, complex III and complex V. Representative immunoblots for steady-state levels of proteins are shown on top (A). The quantitative analyses of the bands by densitometry are shown in B, C, D and E for mitochondrial complex I, complex II, complex III and complex V, respectively. Results shown are fold increases from control from 4 independent experiments compared with control cells. *p<0.05 vs. control. **p<0.01 vs.control.

FIG. 3 shows expression of mitochondrial DNA. 3T3-L1 adipocytes were treated for 48 hrs with hydroxytyrosol. PCR products were quantified fluorescence using SYBR Green. Quantitative values tabulated for D-loop:18sRNA ratio. Results are expressed as fold increase of control. Data are mean±SE (n=5). *P<0.05 vs. control; ** p<0.01 vs. control.

FIG. 4 shows oxygen consumption in 3T3-L1 adipocytes. Equal volumes of cells were separated into aliquots in wells of a 96-well BD Oxygen Biosensor plate. Plates were covered and fluorescence in each well was recorded over time with a fluorescence microplate spectrophotometer. A) Representative oxygen consumption curves. B) Quantitative changes in the respiratory rate of adipocytes during each condition were calculated by determining the kinetic measurements. Values are mean±SE; results shown are fold increases of control from 3 independent experiments compared with control cells. *p<0.05 vs. control.

FIG. 5 shows the effect of treatment with hydroxytyrosol on activities of complexes in adipocytes. (A) Complex I, (B) Complex II, and (C) Complex III. Adipocytes were treated with different concentrations of hydroxytyrosol for 48 hrs. Values are mean±SE of data from three separate experiments for complex I, and six separate experiments for complex II and III, and each experiment was performed in duplicate.*p<0.05, **p<0.01 vs. control.

PGC1α, Peroxisome proliferation activator receptor (PPAR) gamma-coactivator 1 alpha, a transcription coactivator, functions as a master regulator of a wide array of metabolic and physiological processes and is an essential factor in the process of mitochondrial biogenesis. PGC-1α overexpression stimulates mitochondrial biogenesis in 3T3 cells, as shown by increased mitochondrial mass and activity.

Preferably hydroxytyrosol is the only active anti-aging ingredient in the composition.

Thus, the composition according to the present invention and hydroxytyrosol itself keeps one young and/or healthy, brings the anti-aging solution, and prevents the aging process and/or makes one happy or more content while aging.

Another aspect of this invention is the use of a composition comprising hydroxytyrosol for the manufacture of a composition for reducing the prevalence of age-related ailments at a given age in animals, and thereby increasing the likelihood to live longer; for delaying optical signs of the aging process in animals, such as but not limited to hair graying, wrinkles, loss of hearing function, loss of muscle mass, loss of bone density and loss of proper cardiac function; and/or for reducing the risk of lifestyle diseases in animals, which accelerate the ageing process; wherein the composition does essentially not comprise resveratrol.

One object of the present invention is the use of a composition comprising hydroxytyrosol as anti-aging agent, wherein the composition does essentially not comprise resveratrol and wherein the composition is administered orally to animals.

Hydroxytyrosol (3,4-dihydroxyphenylethanol) may be of synthetic origin or it may be isolated from extracts of olive leaves, olive fruits and vegetation water of olive oil production.

Thus, the term “hydroxytyrosol” also encompasses any material or extract of a plant or any material or extract of parts of a plant or any extract/concentrate/juice of fruits of a plant (such as olives) containing it, especially in an amount of at least 30 weight %, preferably in an amount of at least 40 weight-%, more preferably in an amount of at least 50, 55, 60, 65, 70, 75, 80, 85, 90 weight-%, most preferably in an amount of at least 50 weight-%, based on the total weight of the plant material or extract. The terms “material of a plant” and “plant material” used in the context of the present invention means any part of a plant, also the fruits.

In further embodiments of the present invention, hydroxytyrosol derivatives such as esters and physiologically/pharmaceutically acceptable salts may be used instead of or in addition to hydroxytyrosol. Suitable derivatives are known to the person skilled in the art.

Preferred esters of hydroxytyrosol are e.g. acetates or glucuronide conjugates; oleuropein being the most preferred one.

“Essentially not comprising resveratrol” means that the amount of resveratrol in the composition is ≦1 weight-%, preferably ≦0.5 weight-%, more preferably ≦0.1 weight-%, based on the total weight of the composition. It also means that resveratrol is not added intentionally to the composition. Resveratrol may only be in the composition as by-product of a hydroxytyrosol extract/concentrate obtained from plants or fruit of plants such as olives. If present at all, it is at a concentration where its biological activity is not significant.

“The composition is administered orally to animals.” means that the composition is in any form that can be eaten or drunk by animals or put into the stomach of animals via the mouth/jaw.

Thus, the composition is preferably selected from the group of dietary supplements, food additives, functional food, feed additives, functional feed, food premixes, feed premixes, and beverages. Examples of forms of dietary supplements are tablets, pills, granules, dragées, capsules, instant drinks and effervescent formulations.

Examples of food/feed additives are any composition/formulation added to food/feed during its manufacture or its preparation for consumption.

Examples of functional food are dairy products (yoghurts), cereal bars and bakery items such as cakes, cookies, and bread. Clinical nutrition is also encompassed.

Examples of functional feed including pet food compositions are feed intended to supply necessary dietary requirements, as well as treats (e.g., dog biscuits) or other feed supplements. The animal feed comprising the composition according to the invention may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

Examples of food premixes are premixes for manufacture of dairy products, cereal bars, and bakery items such as cakes and cookies, and soups.

A further aspect of the invention relates to a feed additive or additive composition, such as to be added to one or more edible feed substance (s) or ingredient (s), for example to prepare a feed composition or for supplementation to an existing feed to form a feed composition.

The so-called premixes are examples of animal feed additives of the invention. A premix designates a preferably uniform mixture of one or more micro-ingredients with diluent and/or carrier. Premixes are used to facilitate uniform dispersion of micro-ingredients in a larger mix. The premix may be in the form of granules or pellets.

In a particular embodiment, hydroxytyrosol, in the form in which it is added to the food, feed, or when being included in a feed additive, is well-defined. The term well-defined means that the hydroxytyrosol preparation is at least 40% pure. In other particular embodiments the well-defined hydroxytyrosol preparation is at least 60, 65, 70, 75, 80, 85, 88, 90, 92, 94, or at least 95% pure.

Usually fat- and water-soluble vitamins, as well as trace minerals form part of a so-called premix intended for addition to the feed, whereas macro minerals are usually separately added to the feed.

Further, optional, feed-additive ingredients are coloring agents, e.g. carotenoids such as beta-carotene, astaxanthin, and lutein; aroma compounds; stabilisers; antimicrobial peptides; reactive oxygen generating species; and/or at least one enzyme selected from amongst phytase (EC 3.1.3.8 or 3.1.3.26); xylanase (EC 3.2.1.8); galactanase (EC 3.2.1.89); alpha-galactosidase (EC 3.2.1.22); protease (EC 3.4., phospholipase A1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Beverages encompass non-alcoholic and alcoholic drinks as well as liquid preparations to be added to drinking water and liquid food. Non-alcoholic drinks are e.g. instant drinks, soft drinks, sport drinks or sport beverages in general, fruit juices such as e.g. orange juice, apple juice and grapefruit juice; vegetable juices such as tomato juice; smoothies, lemonades, functional water, near-water drinks (i.e. water based drinks with a low calorie content), teas and milk based drinks. Alcoholic drinks are especially beer. Liquid foods are e.g. soups and dairy products (e.g. muesli drinks).

The dietary supplements according to the present invention may further contain protective hydrocolloids, binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Alternatives to dietary supplements which may also be used and are encompassed by the present invention are pharmaceutical compositions.

Beside a pharmaceutically acceptable carrier and hydroxytyrosol (derivatives) with the preferred purity (and further preferences) as given above, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral administration.

The dietary supplements and the pharmaceutical compositions according to the present invention may be in any galenic form that is suitable for oral administration to the animal body (preferably the human body), e.g. in solid form such as tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules. The dietary and pharmaceutical compositions may be in the form of controlled (delayed) release formulations.

According to the present invention such compositions are used for retarding aging processes in animals (preferably in humans), for improving age-related physiological deficits in animals (preferably in humans) and/or for promoting a healthy aging in animals (preferably in humans).

Thus, the present invention is also directed to a composition (with the forms and preferences as given above) which is orally administered to animals (preferably humans) comprising hydroxytyrosol for retarding aging processes in animals (preferably humans), for improving age-related physiological deficits in animals (preferably humans) and/or for promoting a healthy aging in animals (preferably humans), wherein the composition does essentially not comprise resveratrol.

Furthermore, the present invention is directed to a method of retarding aging processes in animals, for improving age-related physiological deficits in animals and/or for promoting a healthy aging in animals by administering to said animal an effective amount of hydroxytyrosol or an effective amount of a composition comprising hydroxytyrosol, wherein the composition does essentially not comprise resveratrol.

Animals in the context of the present invention include humans and encompass mammals, fish and birds. Preferred “animals” are humans, pet animals and farm animals. Especially preferred animals are humans.

Examples for pet animals are dogs, cats, birds, aquarium fish, guinea pigs, (jack) rabbits, hares and ferrets. Examples for farm animals are aquaculture fish, pigs, horses, ruminants (cattle, sheep and goat) and poultry.

“Retarding aging processes in animals” in the context of the present invention means any of:
reducing the prevalence of age-related ailments at a given age, and thereby increasing the likelihood to live longer;
delaying optical signs of the aging process, such as but not limited to hair graying, wrinkles, loss of hearing function, loss of muscle mass, loss of bone density and loss of proper cardiac function; and/or
reducing the risk of lifestyle diseases which accelerate the ageing process.

“Improving age-related physiological deficits in animals” in the context of the present invention means reducing the (average) risk of developing age-related ailments (at a given age).

“Promoting a healthy aging in animals” in the context of the present invention means increasing the healthy life expectancy, i.e. increasing the chance to stay healthy longer.

Such effects are best studied in intervention trials, comparing the average status of aging/disease prevalence in treated individuals versus a non-treated control group.

The daily dosage of hydroxytyrosol for humans (70 kg person) may be at least 0.1 mg. It may vary from 5 to 500 mg, preferably from 10 to 100 mg.

The preferred dose of hydroxytyrosol varies from 0.28 to 1.9 mg/kg metabolic body weight for mammals, whereby
“metabolic body weight” [in kg]=(body weight [in kg])^{0.75}
   for mammals. This means e.g. that for a human of 70 kg the preferred daily dose would vary between 6.77 and 45.98 mg, for a 20 kg dog the preferred daily dose would vary between 2.23 and 15.1 mg.

The invention is now further illustrated by the following, non-limiting examples.

### EXAMPLES

### Example 1

### Soft Gelatin Capsule

Soft gelatin capsules are prepared by conventional procedures providing a dose of hydroxytyrosol of 50 mg per capsule. A suitable daily dose is 1 to 5 capsules.

Other ingredients: glycerol. Water, gelatine, vegetable oil

### Example 2

### Hard Gelatin Capsule

Hard gelatin capsules are prepared by conventional procedures providing a dose of hydroxytyrosol of 75 mg per capsule. A suitable daily dose is 1 to 5 capsules.

Other Ingredients:

Fillers: lactose or cellulose or cellulose derivatives q.s.
Lubricant: magnesium stearate if necessary (0.5%)

### Example 3

### Tablet

Tablets are prepared by conventional procedures providing as active ingredient 100 mg of hydroxytyrosol per tablet, and as excipients microcrystalline cellulose, silicone dioxide (SiO₂), magnesium stearate, crosscarmellose sodium ad 500 mg.

### Example 4

### Soft Drink

A soft drink containing hydroxytyrosol may be prepared as follows:

| | |
|---|---|
| Ingredient | [g] |

A. juice concentrates and water soluble flavours

| | |
|---|---|
| 60.3°Brix, 5.15% acidity | 657.99 |
| 43.5° Brix, 32.7% acidity | 95.96 |
| Orange flavour, water soluble | 3.43 |
| Apricot flavour, water soluble | 6.71 |
| Water | 26.46 |

B. color

| | |
|---|---|
| β-carotene 10% CWS | 0.89 |
| Water | 67.65 |

C. Acid and antioxidant

| | |
|---|---|
| Ascorbic acid | 4.11 |
| Citric acid anhydrous | 0.69 |
| Water | 43.18 |

D. stabilizers

| | |
|---|---|
| Pectin | 0.20 |
| Sodium benzoate | 2.74 |
| Water | 65.60 |

E. oil soluble flavours

| | |
|---|---|
| Orange flavour, oil soluble | 0.34 |
| Orange oil distilled | 0.34 |

F. active ingredient

| | |
|---|---|
| Hydroxytyrosol | Amount providing 15 mg |

Fruit juice concentrates and water soluble flavours are mixed without incorporation of air. The color is dissolved in deionized water. Ascorbic acid and citric acid are dissolved in water. Sodium benzoate is dissolved in water. The pectin is added under stirring and dissolved while boiling. The solution is cooled down. Orange oil and oil soluble flavours are premixed. The active ingredient as mentioned under F is stirred into the fruit juice concentrate mixture of A.

In order to prepare the soft drinks all components A-F are mixed together before homogenizing using a Turrax and then a high-pressure homogenizer (p₁=200 bar, p₂=50 bar).

### Example 5

### Mitochondrial Biogenesis

Anti-rabbit PGC-1α and anti-rabbit PPAR-γ were purchased from Santa Cruz (Calif., USA); anti-α-tubulin from Sigma (St. Louis, Mo., USA); Mito-Tracker Green FM, anti-oxidative complex I, II, III, and V from Invitrogen (Carlsbad, USA); SYBR® GREEN PCR Master Mix from ABI (Warrington, UK); BD Oxygen Biosensor System plate from BD Biosciences (Calif., USA); Hydroxytyrosol (DSM Nutritional Products); Mitochondrial D-loop and 18SRNA primers were synthesized by Bioasia Biotech (Shanghai, China), other reagents for cell culture were from Invitrogen (Carlsbad, USA).

### Cell Culture and Adipocyte Differentiation

Murine 3T3-L1 pre-adipocytes (American Type Culture Collection) were cultured in Dulbecco's Modified Eagle's Medium (DMEM) supplemented with 10% fetal bovine serum and allowed to reach confluence. Differentiation of pre-adipocytes was initiated with 1 μM insulin, 0.25 μM dexamethasone and 0.5 mM 3-isobutyl-1-methylxanthine in DMEM, supplemented with 10% fetal bovine serum. After 48 h, the culture medium was replaced with DMEM supplemented with 10% fetal bovine serum and 1 μM insulin. The culture medium was changed every other day with DMEM containing 10% fetal bovine serum. Cells were used 9-10 days following differentiation induction when exhibiting 90% adipocyte phenotype.

### Determination of Mitochondrial Mass

Adipocytes were trypsinized and centrifuged at 1,000 rpm at 4° C. for 5 min, resuspended in Kreb's Ringer solution buffered with HEPES and 0.1% BSA, then incubated with 0.1 μM MitoTracker Green FM in DMEM for 30 min at 37° C. Cells were centrifuged at 1,000 rpm at 4° C. for 5 min and resuspended in 400 μl of fresh Kreb's Ringer solution buffered with HEPES. To examine relative mitochondrial staining in the fractions, 20×10³ Mitotracker-labeled cells in 200 μl PBS from each fraction were loaded into a 96-well plate and relative fluorescence intensity was read (excitation 485±25 nm; emission 538±25 nm) using a fluorescence microplate spectrophotometer (Molecular probe). Results are expressed as fold increase of the fluorescence intensity over untreated control cells. Values are mean±SE of the results from four independent experiments.

### Western Blot Analysis

After treatment, cells were washed twice with ice-cold PBS, lysed in sample buffer (62.5 mM Tris-Cl pH 6.8, 2% SDS, 5 mM DTT) at room temperature and vortexed. Cell lysates were then boiled for 5 minutes and cleared by centrifugation (13,000 rpm, 10 minutes at 4° C.). Protein concentration was determined using the Bio-Rad DC protein assay. The soluble lysates (10 μg per lane) were subjected to 10% SDS-PAGE, proteins were then transferred to nitrocellulose membranes and blocked with 5% non-fat milk/TBST for 1 h at room temperature. Membranes were incubated with primary antibodies directed against PPAR-γ (1:1000), PGC-1α (1:1000), α-tubulin (1:10 000), Complex I (1:2000), Complex II (1:2000), Complex III (1:2000) and Complex V (1:2000) in 5% milk/TBST at 4° C. overnight. After washing membranes with TBST three times, membranes were incubated with horseradish peroxidase-conjugated secondary antibody for 1 h at room temperature. Western blots were developed using ECL (Roche Manheim, Germany) and quantified by scanning densitometry.

### Measurement of Respiration in Adipocytes

Oxygen consumption by intact cells was measured as an indication of mitochondrial respiration activity. The BD™ Oxygen Biosensor System (BD Biosciences, Franklin Lakes, N.J., USA) is an oxygen sensitive fluorescent compound (tris 1,7-diphenyl-1,10 phenanthroline ruthenium (II) chloride) embedded in a gas permeable and hydrophobic matrix permanently attached to the bottom of a multiwell plate. The concentration of oxygen in the vicinity of the dye is in equilibrium with that in the liquid media. Oxygen quenches the dye in a predictable concentration dependent manner. The amount of fluorescence correlates directly to the rate of oxygen consumption in the well, which in turn can relate to any sort of reaction that can be linked to oxygen consumption. The unique technology allows homogenous instantaneous detection of oxygen levels. After treatment, adipocytes were washed in KRH buffer plus 1% BSA. Cells from each condition were divided into aliquots in a BD Oxygen Biosensor System plate (BD Biosciences) in triplicate. Plates were sealed and “read” on a Fluorescence spectrometer (Molecular probes) at 1-minute intervals for 60 minutes at an excitation wavelength of 485 nm and emission wavelength of 630 nm. The number of cells contained in equal volumes was not statistically significantly between conditions (Wilson-Fritch et al., 2004 J Clin Invest 114:1281-1289).

### Measurement of Mitochondrial DNA

Quantitative PCR was performed in Mx3000P Real-Time PCR system (Stratagene). Reactions were performed with 12.5 μl SYBR-Green Master Mix (ABI), 0.5 μl of each primer (10 μM), 100 ng template (DNA) or no template (NTC), and RNase-free water was added to a final volume of 25 μl. The cycling conditions were as follows: 50° C. for 2 min, initial denaturation at 95° C. for 10 min, followed by 40 cycles of 95° C. for 30 sec, 55° C. for 1 min and 72° C. for 30 sec. Each quantitative PCR was performed in triplicate. The following primers were used:

| | |
|---|---|
| mitochondrial D-loop forward, | |
| 5′-AATCTACCATCCTCCGTG-3′ | (SEQ.ID.NO: 1) |
| reverse | |
| 5′-GACTAATGATTCTTCACCGT | (SEQ. ID.NO: 2) |
| 18SRNA forward: | |
| 5′-CATTCGAACGTCTGCCCTATC-3′ | (SEQ.ID.NO: 3) |
| and | |
| reverse: | |
| 5′-CCTGCTGCCTTCCTTGGA-3′ | (SEQ.ID.NO: 4) |

The mouse 18S rRNA gene served as the endogenous reference gene. The melting curve was done to ensure specific amplification. The standard curve method was used for relative quantification. The ratio of mitochondrial D-loop to 18S rRNA was then calculated. Final results are presented as percentage of control.

### Assays for Activities of Mitochondrial Complex I, II, and III

Adipocytes were cultured in 100 mm plates, washed in PBS, resuspended in an appropriate isotonic buffer (0.25 M sucrose, 5 mM Tris-HCl, pH 7.5, and 0.1 mM phenylmethylsulfonyl fluoride), and homogenized. Mitochondria were isolated by differential centrifugation of the cell homogenates. NADH—CoQ oxidoreductase (Complex I), succinate-CoQ oxidoreductase (complex II), CoQ-cytochrome c reductase (complex III) were assayed spectrometrically using the conventional assays (Picklo and Montine, 2001 Biochim Biophys Acta 1535: 145-152; Humphries, K. M., and Szweda, L. I. 1998 Biochemistry 37:15835-15841), with minor modifications.

### Statistical Analysis

All qualitative data were representative of at least three independent experiments. Data are presented as means±SE. Statistical significance was determined by using one-way ANOVA with Bonferroni's post hoc tests between the two groups. The criterion for significance was set at p<0.05.

### Results:

### Effect of Hydroxytyrosol on PGC-1α Protein Level in Adipocytes

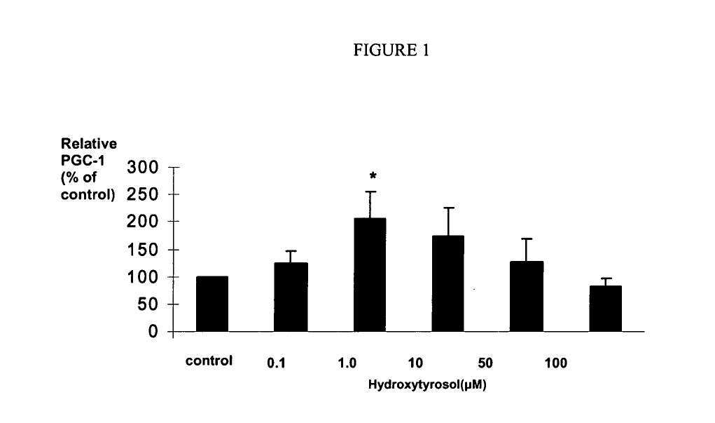

As shown in FIG. 1, hydroxytyrosol showed a bell-shape effect on increasing PGC-1α from 0.1 to 10.0 μM with a maximum protein expression at 1.0 μM (205±52%, p, 0.05 vs.control).

### Effect of Hydroxytyrosol on Complex I, II, III and V Protein Expression in Adipocytes

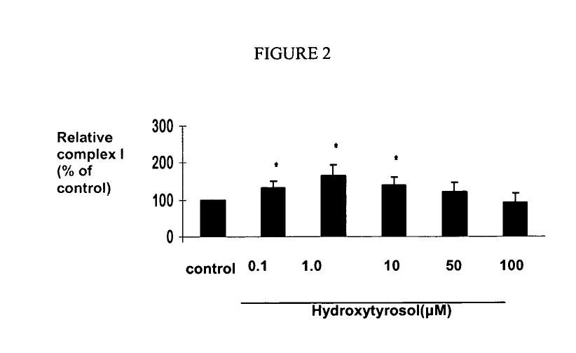

Mitochondrial complexes were determined by Western blotting (FIG. 2). An increase on mitochondrial electron transport complex I protein was observed with hydroxytyrosol treatment at 0.1 μM (131±16%, p<0.05 vs.control), 1.0 μM (163±31%, p<0.01 vs.control) and 10.0 μM (138±21%, p<0.05 vs.control).

### Effects of Hydroxytyrosol on Mitochondrial DNA

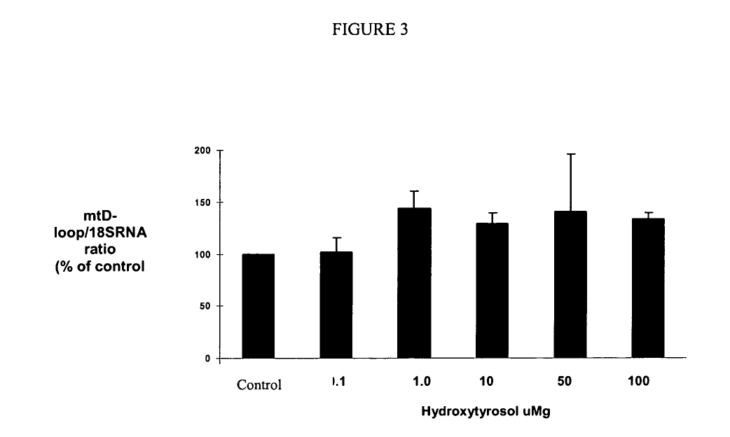

As the D-loop is known as the major site of transcription initiation on both the heavy and light strands of mtDNA, we examined in vitro whether hydroxytyrosol could increase mtDNA expression. As shown in FIG. 3, the ratio of mt D-loop/18SRNA was significantly increased in adipocytes treated with hydroxytyrosol at 1.0 μM.

### Effect of Hydroxytyrosol on Oxygen Consumption in Adipocytes

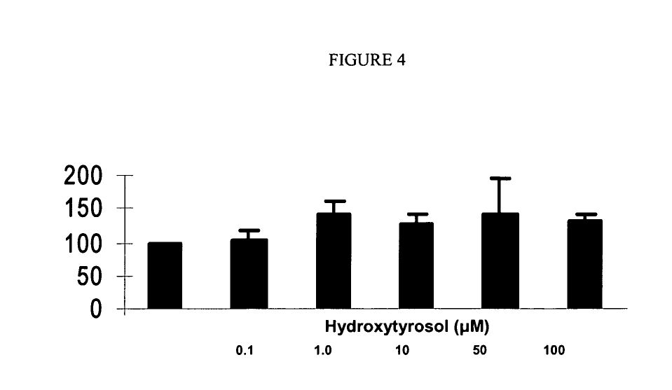

To determine whether increased mitochondrial biogenesis is accompanied by changes in oxygen consumption, cells were treated with hydroxytyrosol at 0.1-10 μM. As shown in FIG. 4, the basal rate of oxygen consumption was significantly increased in adipocytes treated with hydroxytyrosol at 1.0-5.0 μM.

### Assays for Activities of Mitochondrial Complex I, II, and III

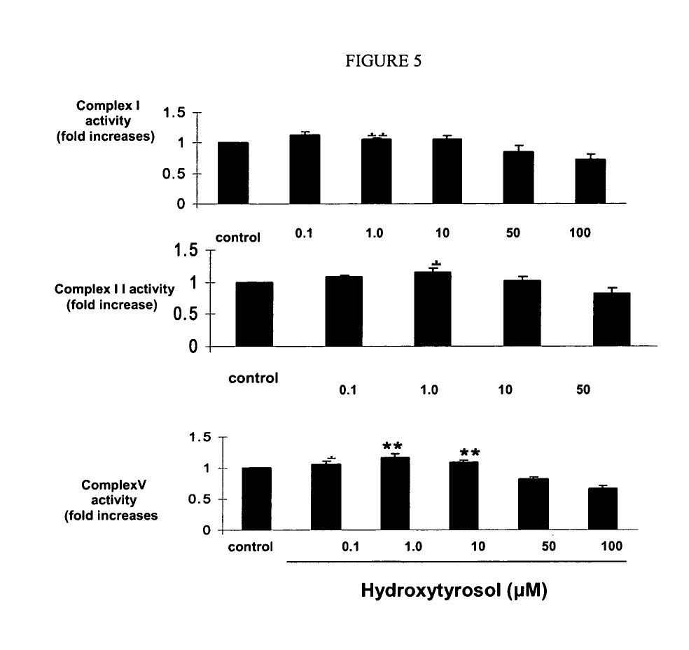

Hydroxytyrosol showed significant increase in the activity of mitochondrial complex I and II in adipocytes cells at 0.1 μM and 1.0 μM respectively relative to control (FIGS. 5A and 5B). Hydroxytyrosol also showed significant increase in the activity of mitochondrial complex III in adipocytes cells at 0.1 μM-10 μM.

The data show that hydroxytyrosol increases expression of mtDNA-encoded polypeptides and mitochondrial electron transport complex I. Moreover the activity of complex, II and V and oxygen consumption is increased, thus leading to an increase in mitochondrial respiratory activity. Finally hydroxytyrosol increased the expression of the peroxisome proliferator-activated receptor-γ coactivator 1 (PGC-1) which is implicated in the control of mitochondrial activity and mitochondrial biogenesis. This increase in PGC-1 leads to an increased mitochondrial biogenesis.

In summary hydroxytrsol promotes mitochondrial activity and such can be used to prevent or treat the ageing process.

1. Use of a composition comprising hydroxytyrosol as anti-aging agent, wherein the composition does essentially not comprise resveratrol and wherein the composition is administered orally to animals.

2. Use of a composition comprising hydroxytyrosol for (the manufacture of a composition for) retarding aging processes in animals, for improving age-related physiological deficits in animals and/or for promoting a healthy aging in animals, wherein the composition does essentially not comprise resveratrol.

3. Use of a composition comprising hydroxytyrosol for (the manufacture of a composition for) reducing the prevalence of age-related ailments at a given age in animals, and thereby increasing the likelihood to live longer; for delaying optical signs of the aging process in animals, such as but not limited to hair graying, wrinkles, loss of hearing function, loss of muscle mass, loss of bone density and loss of proper cardiac function; and/or for reducing the risk of lifestyle diseases in animals, which accelerate the ageing process; wherein the composition does essentially not comprise resveratrol.

4. The use according to claim 1, wherein hydroxytyrosol is the only active anti-aging ingredient in the composition.

5. The use according to claim 1, wherein the composition is selected from the group of dietary supplements, food additives, functional food, food premixes, feed additives, functional feed, feed premixes, and beverages.

6. Composition which is orally administered to animals comprising hydroxytyrosol for retarding aging processes in said animals, for improving age-related physiological deficits in said animals and/or for promoting a healthy aging in said animals, wherein the composition does essentially not comprise resveratrol.

7. Composition which is orally administered to animals comprising hydroxytyrosol for reducing the prevalence of age-related ailments at a given age, and thereby increasing the likelihood to live longer; for delaying optical signs of the aging process, such as but not limited to hair graying, wrinkles, loss of hearing function, loss of muscle mass, loss of bone density and loss of proper cardiac function; and/or for reducing the risk of lifestyle diseases, which accelerate the ageing process; wherein the composition does essentially not comprise resveratrol.

8. The composition according to claim 6, wherein the composition is in form of a dietary supplement, a food additive, a functional food, a feed additive, a functional feed or a beverage.

9. The composition according to claim 6, wherein the animals are humans.

10. Method of retarding aging processes in animals, for improving age-related physiological deficits in animals and/or for promoting a healthy aging in animals by administering to said animal an effective amount of hydroxytyrosol or an effective amount of a composition comprising hydroxytyrosol, wherein the composition does essentially not comprise resveratrol.

11. Method of reducing the prevalence of age-related ailments at a given age in animals, and thereby increasing the likelihood to live longer; of delaying optical signs of the aging process in animals, such as but not limited to hair graying, wrinkles, loss of hearing function, loss of muscle mass, loss of bone density and loss of proper cardiac function; and/or of reducing the risk of lifestyle diseases in animals, which accelerate the ageing process by administering to said animal an effective amount of hydroxytyrosol or an effective amount of a composition comprising hydroxytyrosol, wherein the composition does essentially not comprise resveratrol.

12. The method according to claim 10, wherein the animals are humans.
